(19) United States
(12) Patent Application Publication
(10) Pub. No.: US 2006/0229539 A1
(43) Pub. Date: Oct. 12, 2006

(54) Therapeutic tool for tennis elbow

(76) Inventor: Yoshitaka Toda, Osaka (JP)

Correspondence Address: BIRCH STEWART KOLASCH & BIRCH, PO BOX 747, FALLS CHURCH, VA, 22040-0747, US

(21) Appl. No.: 11/398,562

(22) Filed: Apr. 6, 2006

(30) Foreign Application Priority Data
Apr. 8, 2005 (JP) 2005-112055

(51) Int. Cl. A61F 5/00

(52) U.S. Cl. 602/21

(57) ABSTRACT

A therapeutic tool 1 for tennis elbow has a wrist holding part 2 and a finger holding part 3. The wrist holding part 2 is extendable in a longitudinal direction The wrist holding part 2 has an end 22 and an end 23 which are pulled to wind around a wrist. The end 22 and the end 23 are attached to a pile part 21 on the other end. The finger holding part 3 has a shape of a belt and extendable in a longitudinal direction. The finger holding part 3 is diverged at a diverging part 33. The diverging part 33 is adjusted to a base of a dorsal side of a finger. An end 31 and an end 32 are pulled to fold back at both sides of the base of the finger and attached to the wrist holding part 2.

This application claims priority on Patent Application No. 2005-112055 filed in JAPAN on Apr. 8, 2005. The entire contents of this Japanese Patent Application are hereby incorporated by reference.

### BACKGROUND OF THE INVENTION

1. Field of the Invention

The present invention relates to a therapeutic tool for treatment and prevention of tennis elbow.

2. Description of the Related Art

Tennis elbow is an insertion disorder which appears around origin of wrist and finger extensor muscles to a humeral lateral epicondyle. This disorder is caused by repetitive very little external force. Tennis elbow, as its name suggests, previously had been considered to be caused by strong mechanical load applied on a wrist joint when doing sports such as tennis.

Tennis elbow also frequently affects people such as homemakers and office workers who are not involved in heavy work. The incidence of tennis elbow caused by doing sports such as golf or tennis is only 30.8%. This is less than the incidence of 47.6% by doing office work and the like.

One of the causes of tennis elbow is tension of muscles by operating a keyboard or a mouse. In the present day, the use of computers has become widespread and many people are working most of the day with computers. When operating a keyboard or a mouse, fingers are extended and extensor digitorum communis muscles are used with high frequency.

For treatment of tennis elbow, a band is put around an affected elbow. Strapping the elbow with the band to control movements of the elbow speeds recovery of the elbow. For treatment of tennis elbow, the band is also put around a wrist to control the movements of the wrist.

Bracing the elbow and the wrist by putting a band around the elbow or the wrist is not sufficient to recover from tennis elbow.

As mentioned above, operating a keyboard or a mouse causes tennis elbow. The inventor consequently considers that operating the keyboard or the mouse and the like requires fingers and wrist joint to extend, and thereby the load on the humeral lateral epicondyle is increased to produce pain. The inventor carried out experiments wherein electrodes of electromyograph were set around origin of extensor carpi radialis brevis (humeral lateral epicondyle) to obtain electric potential when wrist joint was dorsiflexed and fingers (index finger, middle finger, ring finger and little finger) were extended, and amplitude of electric potential was measured.

The experiment was intended for 24 subjects comprising 10 examples of healthy subjects (average age: 37.6±14.5) who had never had a pain in humeral lateral epicondyle before, and 14 examples of patients with tennis elbow (average age: 45.1±6.3). For statistical test, chi-square test and one-way analysis of variance were used. Rectified average electric activity was measured with a portable type electromyograph (Muscle Analyzer, available from MP JAPAN CO., LTD). When measuring, an anode was set on 2 cm distal to origin of extensor carpi radialis brevis (humeral lateral epicondyle), and a cathode was set on another 2 cm distal to the anode. The patients with tennis elbow were set the electrodes on affected side, and all the healthy subjects were set the electrodes on their dominant arm because it is reported that tennis elbow overwhelmingly affects dominant arm.

When measuring with electromyograph, the load was applied as follows: 1) dorsiflexion of wrist: all the finger joints are lightly flexed (in a relaxed state) with 0 degree of flexion and dorsiflexion of wrist joint, and then the wrist joint was dorsiflexed to a maximum extent. This state was kept for 10 seconds. 2) Extension of fingers (index finger, middle finger, ring finger and little finger): from the state of 0 degree of flexion and dorsiflexion of wrist joint, only metacarpophalangeal joint was extended to a maximum extent (distal interphalangeal joint and proximal interphalangeal joint were with flexion of 0 degree), and this state was kept for 10 seconds. In addition, to prevent the action of metacarpophalangeal joint of the other fingers, assistants restrained extension of the other fingers with a plastic ruler when measuring each of the fingers.

Integral values of the rectified electric potential (the sum of action potential for 10 seconds) were obtained under said state wherein the load was applied for 10 seconds. By obtaining the sum of the action potential, each of the loads applied on humeral lateral epicondyle at each motion can be calculated. The load was applied 3 times for each motion, and its average value was considered to be the measuring result of each patient.

FIG. 9 is a graph which shows the sum of action potential obtained when a wrist joint and all the fingers are extended. As the FIG. 9 shows, 15 examples of the 24 examples (62.5%) have the highest electric potential at a middle finger and 9 examples of the 24 examples (37.5%) have the highest electric potential at a wrist joint. There is no example which has the highest electric potential at an index finger, a ring finger and a little finger. As to this tendency, there is no difference between a group of patients with tennis elbow and a group of healthy subjects. When the middle finger is extended, the load applied on humeral lateral epicondyle is greater than when the wrist joint is dorsiflexed, whether patients or healthy subjects. Extensor carpi radialis brevis which is attached to humeral lateral epicondyle as origin, leads to dorsum of hand through the second tube under extensor retinaculum, and reaches the back of the base of metacarpal III. Extension movement of the middle finger increases the load on humeral lateral epicondyle. In view of these results, the inventor considers that controlling of extension of not only wrist joint but also the middle finger decreases the load on humeral lateral epicondyle, and reduces pain by tennis elbow.

An object of the present invention is to provide a therapeutic tool that decreases the load on humeral lateral epicondyle to reduce pain by tennis elbow and prevent the onset of tennis elbow.

### SUMMARY OF THE INVENTION

Therapeutic tool according to the present invention has

(1) a wrist holding part, and

(2) a finger holding part which is extendable in the longitudinal direction and diverging at a diverging part on a designated place, said diverging part being adjusted to a base of a dorsal side of a finger, both heads on the diverging side being pulled to fold back at both sides of said base of the finger, and said both heads being constructed to attach to said wrist holding part.

Preferably, the wrist holding part has a shape of a tape which has one end and the other end, said wrist holding part is extendable in a longitudinal direction, said one end and the other end have hook and loop fasteners, said one end is pulled to wind around a wrist, and said one end is constructed to attach to the other end. Preferably, the both heads on the diverging side of said finger holding part have hook and loop fasteners. Preferably, the other end of said finger holding part which is not diverged is constructed to be freely put on and removed from said wrist holding part.

According to the therapeutic tool for tennis elbow of the present invention, controlling the extension of a wrist and fingers, especially a middle finger decreases the load on humeral lateral epicondyle. Controlling the tension of muscles reduces pain by tennis elbow and prevents the onset of tennis elbow.

### BRIEF DESCRIPTION OF THE DRAWINGS

FIG. 1 is a perspective view illustrating a therapeutic tool for tennis elbow attached to a hand and fingers which are operating a mouse, according to one embodiment of the present invention;

FIG. 2 is a plain view illustrating the therapeutic tool shown in FIG. 1 which is attached to a hand and fingers;

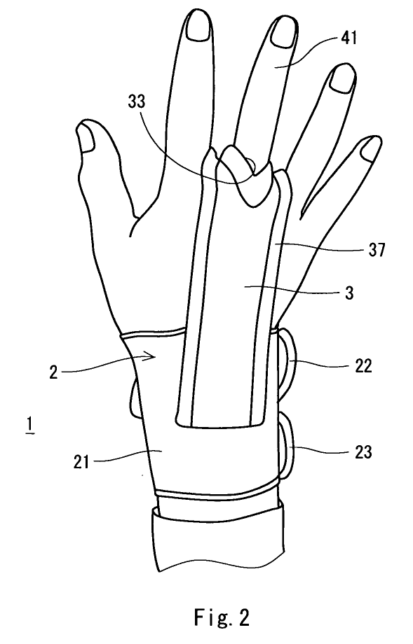

FIG. 3 is a back view of FIG. 2;

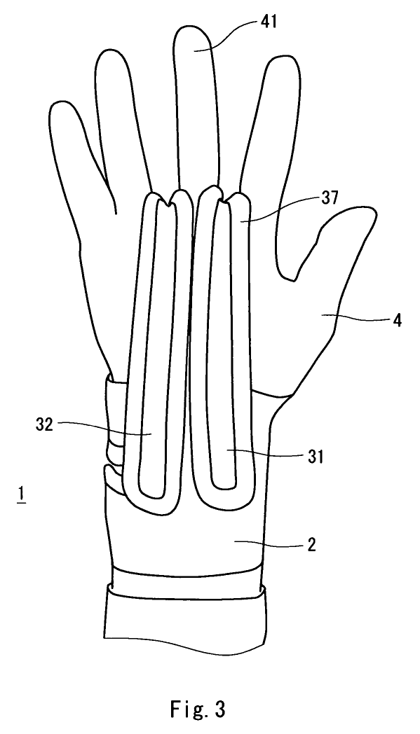

FIG. 4 is a plain view illustrating the therapeutic tool shown in FIG. 1;

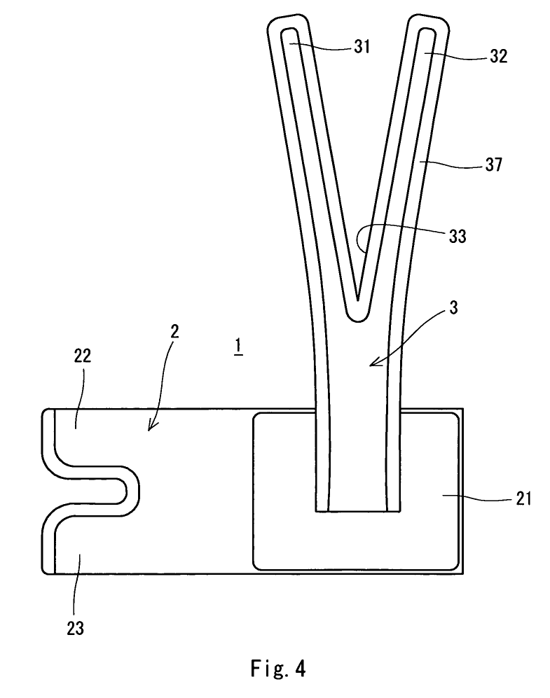

FIG. 5 is a back view illustrating the therapeutic tool shown in FIG. 4;

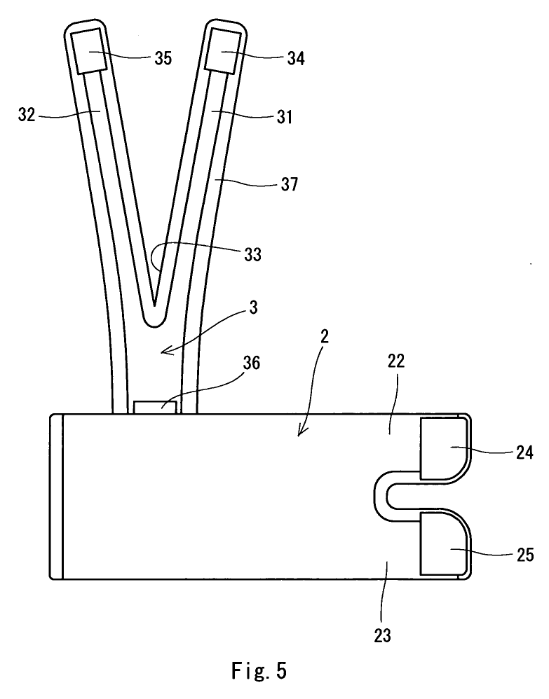

FIG. 6 is a perspective view illustrating the therapeutic tool shown in FIG. 1 which is being worn;

FIG. 7 is a graph showing changes of Visual Analog Scale (VAS);

FIG. 8 is a graph showing changes of difficulty in Ability of Daily Living (ADL); and

FIG. 9 is a graph showing the sum of action potential.

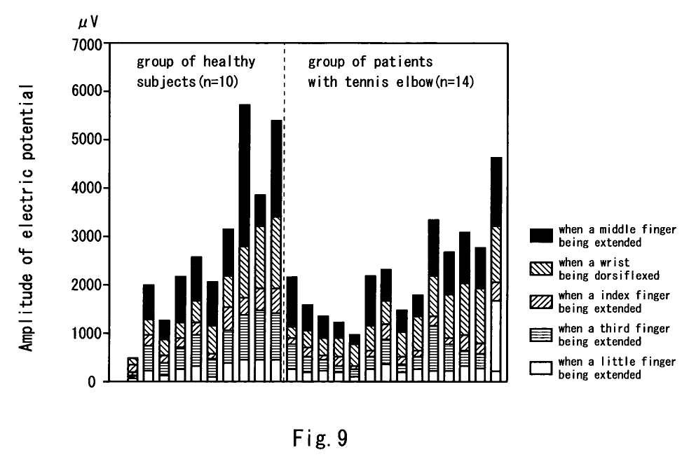

### DESCRIPTION OF THE PREFERRED EMBODIMENTS

The present invention is hereinafter described in detail with appropriate references to the accompanying drawing according to the preferred embodiments.

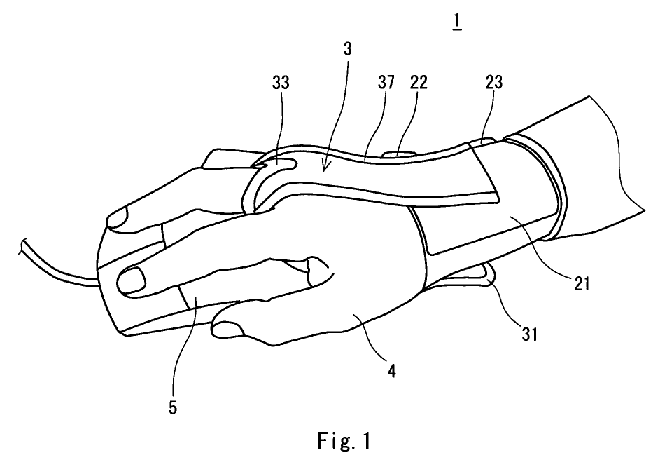

A therapeutic tool 1 for tennis elbow illustrated in FIG. 1 has a wrist holding part 2 and a finger holding part 3. The wrist holding part 2 is rectangular. The wrist holding part 2 has a shape of a tape. The wrist holding part 2 comprises a textile in which rubber thread and the like is woven like mesh. This textile is elastic. This wrist holding part 2 is extendable in a longitudinal direction. One end of the wrist holding part 2 has 2 ends: an end 22 and an end 23 which are diverged upward and downward. The other end of the wrist holding part 2 has a pile part 21 which occupies almost half the area of the wrist holding part 2. The pile part 21 is a hook and loop fastener. The backside of the end 22 and the end 23 is attached a hook part 24 and a hook part 25 respectively. The hook part 24 and the hook part 25 are hook and loop fasteners.

The finger holding part 3 has a shape of a belt. The finger holding part 3 comprises a textile in which rubber thread and the like is woven like mesh. This textile is elastic. The finger holding part 3 is extendable in a longitudinal direction. The finger holding part 3 diverges into two at a diverging part 33. The diverging part 33 is positioned at almost one-third of the length of the finger holding part 3. The diverged side has an end 31 and an end 32 which are attached a hook part 34 and a hook part 35 to the backside thereof respectively. The hook part 34 and the hook part 35 are hook and loop fasteners. The backside of the other end of the finger holding part 3 has a hook part 36. The hook part 36 is a hook and loop fastener. The longitudinal end of the finger holding part 3, the end 31 and the end 32 are hemmed with a bias tape 37.

The therapeutic tool 1 is used as follows. A wearer first hooks the hook part 36 which is attached on the one end of the finger holding part 3, on the pile part 21 of the hand holding part 2. The hook part 36 is hooked at any position on the pile part 21 which suits the wrist size of the wearer. The wearer reverses the therapeutic tool 1 and adjusts the wrist of a hand 4 being affected by tennis elbow to the backside of the wrist holding part 2. The wearer pulls the end 22 and the end 23 on diverging side to wind the wrist holding part 2 around the wrist. The wearer hooks the hook part 24 and the hook part 25 which are attached on the backside of the end 22 and the end 23, on the pile part 21 to fix the wrist holding part 2 to the wrist.

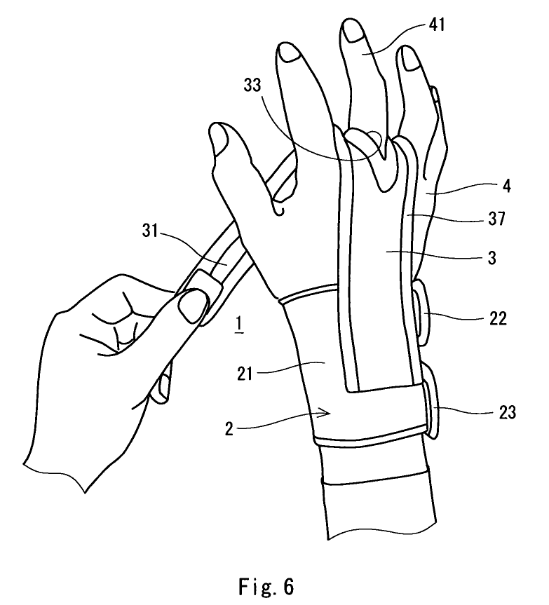

The wearer raises the hand 4 to adjust the diverging part 33 of the finger holding part 3 to a base of a dorsal side of a middle finger 41. The wearer pulls the end 31 and the end 32 on the diverging side with the other hand to fold back the finger holding part 3 at both sides of said base. The wearer pulls the finger holding part 3 across the hand 4 to the palm side and hooks the end 31 and the end 32 on the pile part 21 of the wrist holding part 2 to fix the finger holding part 3. In FIG. 6, the end 31 and the end 32 of the finger holding part 3 are pulled to fold back at both sides of the base of the dorsal side of the middle finger 41. The finger holding part 3 is fastened on the pile part 21 of the wrist holding part 2.

Regarding this therapeutic tool 1, the following is the result of a clinical test which is intended for 24 examples of patients with tennis elbow (average age: 46.6±10.6, 6 male examples and 18 female examples). Among the 24 patients with tennis elbow, persons having an odd numbered birth date (13 examples) were assigned to the therapeutic tool 1 for tennis elbow relating the present invention. Persons having an even numbered birth date (11 examples) were assigned to the conventional band which straps the elbow. After one week, the evaluation of pain was made.

The evaluation of pain was made with Visual Analog Scale (VAS). In this VAS, patients were told, “show your present pain visually assuming the far left means no pain and the far right means extremely painful”. On the line of 100 mm, the length (mm) between the far left and the point which the patients indicated was shown. The difficulty in Ability of Daily Living (ADL) was also measured. The difficulty in ADL was shown with the sum of points which were given to each of the following actions: 1. put on trousers, 2. remove a lid of a plastic bottle, 3. squeeze a wet towel, 4. wipe the floor with a cloth, 5. turn a knob to open a door, 6. carry a bottle of beer with one hand, 7. use a keyboard or a mouse of a personal computer, 8. do sports such as tennis or golf which requires grasping power. When the action was easily done, zero point was given, and when the action was done with difficulty, one point was given.

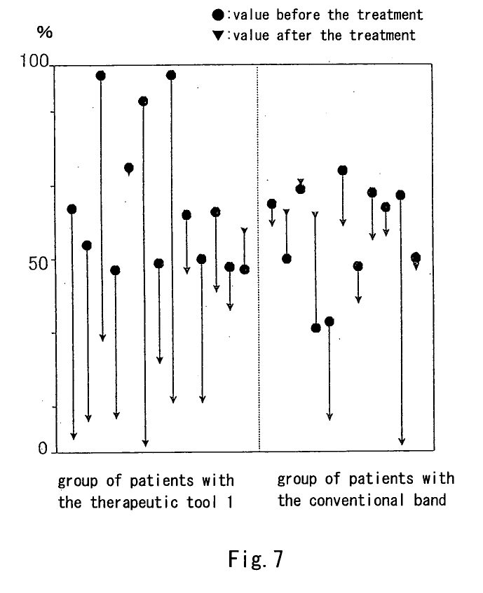

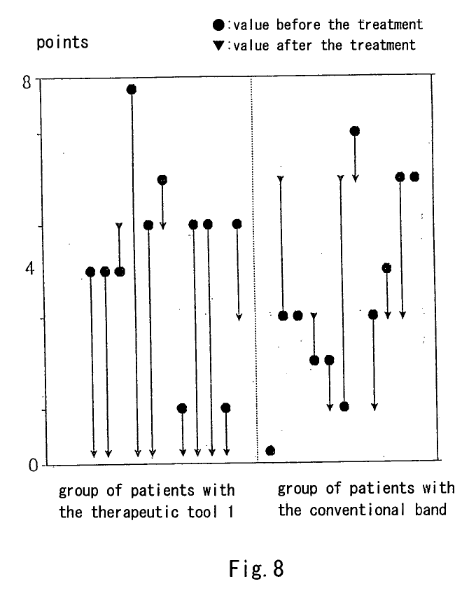

FIG. 7 is a graph showing changes of Visual Analog Scale (VAS) of a group of patients with the therapeutic tool 1 and of a group of patients with the conventional band. FIG. 8 is a graph showing changes of difficulty in Ability of Daily Living (ADL) of the group of patients with the therapeutic tool 1 and of the group of patient with the conventional band. Concerning VAS, the value before the treatment is subtracted from the value after the treatment to obtain a remission rate (minus value means remission). Concerning the difficulty of ADL, the value before the treatment is subtracted from the value after the treatment to obtain remission points (minus value means remission). For statistical test, chi-square test and one-way analysis of variance were used.

Concerning age and sex, there is no significant difference between the group of patients with the therapeutic tool 1 and the group of patient with the conventional band (significant probability p>0.05). The remission rate of VAS shown in the FIG. 7 is −38.5±32.5% in the group of patients with the therapeutic tool 1, and −8.7±24.4% in the group of patients with the conventional band. This shows the pain of the group of patients with the therapeutic tool 1 is remitted significantly compared to the pain of the group of patients with the conventional band (p=0.021).

The remission points of the difficulty of ADL shown in the FIG. 8 is −3.2±2.6 points for the group of patients with the therapeutic tool 1, and −0.1±2.3 points for the group of patients with the conventional band. This shows the pain of the group of patients with the therapeutic tool 1 is remitted significantly compared to the pain of the group of patients with the conventional band, and there is a significant difference between both of the groups (p=0.005).

By wearing the therapeutic tool 1 for tennis elbow, extensor carpi radialis brevis and extensor digitorum communis muscle is passively stretched in daily life. By wearing the therapeutic tool 1, the movement of extensor carpi radialis brevis is passively controlled. It is considered that wearing the therapeutic tool 1 reduces pain by tennis elbow in a short period of one week and enables the patients to resume working with computers. This therapeutic tool 1 effectively solves the problem in the computerized society.

It is reported that the etiology of tennis elbow is tear of extensor carpi radialis brevis having origin at humeral lateral epicondyle caused by repetitive very little external force based on aging. To prevent a recurrence of tennis elbow, exercise therapy by active resistive exercise to extensor carpi radialis brevis is effective after the pain is reduced by wearing the therapeutic tool 1.

The therapeutic tool 1 may be composed by material for swimming wear. This material for swimming wear comprises 95% of polyester and 5% of polyurethane, for example. This material for swimming wear is quick to dry when it gets wet. This material for swimming wear has textile pattern which does not easily stick to the skin and reduces discomfort when it gets wet. By using this material for swimming wear to the therapeutic tool 1, patients with tennis elbow can improve earlier because they do not need to take the therapeutic tool 1 off even when they cook or wash and the like.

As a means of attaching both of the ends of the wrist holding part 2 to each other, a lock part which is like a hook made of metal or synthetic resin may be provided. As a means of attaching the finger holding part 3 to the wrist holding part 2, a lock part which is like a hook made of metal or synthetic resin may be provided. Hook and loop fastener is easy to put on and take off. The wearer can freely change the position where both of the ends of the wrist holding part 2 are to be attached and the position on the wrist holding part 2 where the finger holding part 3 is to be attached, according to the size of the hand 4 of the wearer. In this respect, as a means of attaching both of the ends of the wrist holding part 2 to each other, hook and loop fastener is preferred.

In the therapeutic tool 1, the finger holding part 3 controls the extension of the middle finger 41. The finger holding part 3 may be put over another finger which is easy for the wearer to extend when operating a computer.

The foregoing description is just for illustrative examples, and various modifications can be made in the scope without departing from the principles of the present invention.

What is claimed is:

1. A therapeutic tool for tennis elbow has a wrist holding part, and
a finger holding part which is extendable in the longitudinal direction and diverging at a diverging part on a designated place, said diverging part being adjusted to a base of a dorsal side of a finger, both heads on the diverging side being pulled to fold back at both sides of said base of the finger, and said both heads being constructed to attach to said wrist holding part.

2. The therapeutic tool for tennis elbow according to claim 1 wherein the wrist holding part which has a shape of a tape which has one end and the other end, said wrist holding part is extendable in a longitudinal direction, said one end and the other end have hook and loop fasteners, said one end is pulled to wind around a wrist, and said one end is constructed to attach to the other end.

3. The therapeutic tool for tennis elbow according to claim 1 wherein the both heads on the diverging side of said finger holding part have hook and loop fasteners.

4. The therapeutic tool for tennis elbow according to claim 1 wherein the other end of said finger holding part which is not diverged is constructed to be freely put on and removed from said wrist holding part.
